(19) United States
(12) Patent Application Publication
(10) Pub. No.: US 2025/0269158 A1
(43) Pub. Date: Aug. 28, 2025

(54) PERFUSION DILATATION CATHETER

(71) Applicant: Mavericks Foundation LLC, La Grange, IL (US)

(72) Inventors: Ravi Ramana, La Grange, IL (US); Rahul Sharma, Palo Alto, CA (US)

(73) Assignee: Mavericks Foundation LLC, La Grange, IL (US)

(21) Appl. No.: 18/857,915

(22) PCT Filed: Mar. 22, 2023
(86) PCT No.: PCT/US2023/015851

§ 371 Date: Oct. 18, 2024

Related U.S. Application Data
(60) Provisional application No. 63/363,139, filed on Apr. 18, 2022.

(51) Int. Cl. A61M 29/02; A61F 2/24; A61M 25/10

(52) U.S. Cl. CPC A61M 29/02; A61F 2/2433; A61M 25/10; A61M 2025/1061; A61M 2029/025

(57) ABSTRACT

Perfusion dilatation catheters and methods of use are provided. The perfusion dilatation catheter may comprise an elongated catheter shaft comprising a proximal end and a distal end. The elongated catheter shaft may be configured to enclose a plurality of lumens comprising a proximal balloon inflation lumen and a distal balloon inflation lumen, and may be configured to house at least one inflation port configured to supply at least one inflation medium through the proximal balloon inflation lumen and the distal balloon inflation lumen. The perfusion dilatation catheter may further comprise a proximal balloon system comprising a proximal balloon positioned along the elongated catheter shaft and the proximal balloon inflation lumen, and a distal balloon system comprising a distal balloon positioned along the elongated catheter shaft, distal to the proximal balloon, and the distal balloon inflation lumen.

### CROSS-REFERENCE TO RELATED APPLICATION

The present application claims priority to U.S. Provisional Patent Application No. 63/363,139, filed Apr. 18, 2022, entitled “PERFUSION DILATION CATHETER,” the disclosure of which is herein incorporated by reference in its entirety.

### TECHNICAL FIELD

Embodiments of the present disclosure relate to perfusion dilatation catheter devices and methods of use and, in particular, to perfusion dilatation catheters having proximal and distal balloon systems.

### BACKGROUND

Medical catheters are often used during treatment of cardiovascular disorders. These disorders often require patients to undergo invasive cardiovascular procedures, which can accompany the use of medical catheters.

Due to the delicate nature of the cardiovascular system, the use of medical catheters in the cardiovascular system (e.g., percutaneous structural heart interventions) is an invasive and potentially dangerous procedure. Due to the high stakes of such procedures, it is imperative that medical catheters be configured for accuracy and reproducibility. The greater the accuracy and reproducibility, the greater the chances of improvement in short-and long-term clinical outcomes of cardiovascular patients.

In the current era of structural heart intervention therapy, there are several percutaneous heart valve devices and delivery systems that are typically used. However, these percutaneous structural heart therapies and procedures are limited by adverse outcomes seen in first-generation, second-generation, and third-generation device technologies. For example, current transcatheter therapy for patients with aortic valve disease can be limited by 1) inaccurate assessments of the location of a physicians' devices based on two-dimensional-only fluoroscopic views, and 2) the inability to maintain co-axial alignment of the physicians' equipment to safely and reliably traverse bioprosthetic aortic valves and their stent frames. These limitations may lead to a suboptimal location of the transcatheter heart valve and potentially subsequent adverse outcomes (e.g., paravalvular leak, valve embolization, aortic injury, need for multiple valve implants, need for emergent heart surgery and/or patient death).

For at least these reasons, medical catheters, and methods of use, which improve accuracy of safely, reliably, and efficiently performing cardiovascular procedures are needed. These procedures may comprise, e.g., crossing a bioprosthetic aortic valve before and during valve heart valve procedures (e.g., TAVR (Transcatheter Aortic Valve Replacement) and Valve-in-Valve TAVR procedures).

### SUMMARY

According to an aspect of the present disclosure, a perfusion dilatation catheter is provided. The perfusion dilatation catheter may comprise an elongated catheter shaft comprising a proximal end and a distal end. The elongated catheter shaft may be configured to enclose a plurality of lumens comprising a proximal balloon inflation lumen and a distal balloon inflation lumen, and may be configured to house at least one inflation port configured to supply at least one inflation medium through the proximal balloon inflation lumen and the distal balloon inflation lumen. The perfusion dilatation catheter may further comprise a proximal balloon system including a proximal balloon positioned along the elongated catheter shaft and the proximal balloon inflation lumen, and a distal balloon system comprising a distal balloon positioned along the elongated catheter shaft, distal to the proximal balloon, and the distal balloon inflation lumen.

According to various embodiments, the proximal balloon and the distal balloon may be affixed to an outer surface of the elongated catheter shaft and may be separated by a set distance.

According to various embodiments, the set distance may be approximately 10-15 mm.

According to various embodiments, the at least one inflation port may comprise a first inflation port and a second inflation port, the first inflation port may be configured to supply the at least one inflation medium through the proximal balloon inflation lumen to the proximal balloon, and the second inflation port may be configured to supply the at least one inflation medium through the distal balloon lumen to the distal balloon.

According to various embodiments, the distal balloon may comprise a perfusion-style balloon.

According to various embodiments, the distal balloon may be tapered toward the distal end of the elongated catheter shaft.

According to various embodiments, the distal balloon may have an inflated diameter of approximately 15-20 mm.

According to various embodiments, the proximal balloon may comprise a post-dilatory noncompliant balloon.

According to various embodiments, the proximal balloon may have an inflated diameter of approximately 15-20 mm.

According to various embodiments, the distal balloon may comprise a proximal end and a distal end, and the distal end of the distal balloon may be approximately 8-10 mm from the distal end of the elongated catheter shaft.

According to various embodiments, the plurality of lumens may comprise a lumen configured to receive a curved-tipped guidewire.

According to various embodiments, the plurality of lumens may comprise a plurality of wire lumens. According to various embodiment, each wire lumen, of the plurality of wire lumens, may be configured to receive a wire through the elongated catheter shaft to the distal end of the elongated catheter shaft.

According to another aspect of the present disclosure, a perfusion dilatation catheter is provided. The perfusion dilatation catheter may comprise an elongated catheter shaft comprising a proximal end and a distal end. The elongated catheter may be configured to enclose a plurality of lumens, comprising a proximal balloon inflation lumen and a distal balloon inflation lumen, and may be configured to house at least one inflation port configured to supply at least one inflation medium through the proximal balloon inflation lumen and the distal balloon inflation lumen. The perfusion dilatation catheter may further comprise a distal balloon system comprising a perfusion-style balloon positioned along the elongated catheter shaft and tapered toward the distal end of the elongated catheter shaft and the distal balloon inflation lumen, and a proximal balloon system comprising a post-dilatory noncompliant balloon positioned along the elongated catheter shaft, distal to the perfusion-style balloon and the proximal balloon inflation lumen.

According to various embodiments, the perfusion-style balloon and the post-dilatory noncompliant balloon may be affixed to an outer surface of the elongated catheter shaft, and are separated by a set distance.

According to various embodiments, the set distance may be approximately 10-15 mm.

According to various embodiments, the at least one inflation port may comprise a first inflation port and a second inflation port, the first inflation port may be configured to supply the at least one inflation medium through the proximal balloon inflation lumen to the post-dilatory noncompliant balloon, and the second inflation port may be configured to supply the at least one inflation medium through the distal balloon lumen to the perfusion-style balloon.

According to various embodiments, the perfusion-style balloon may be tapered toward the distal end of the elongated catheter shaft.

According to various embodiments, the perfusion-style balloon may have an inflated diameter of approximately 15-20 mm.

According to various embodiments, the post-dilatory noncompliant balloon may have an inflated diameter of approximately 15-20 mm.

According to various embodiments, the perfusion-style balloon may comprise a proximal end and a distal end, and the distal end of the perfusion-style balloon may be approximately 8-10 mm from the distal end of the elongated catheter shaft.

According to various embodiments, the plurality of lumens may comprise a plurality of wire lumens, each wire lumen being configured to receive a wire through the elongated catheter shaft to the distal end of the elongated catheter shaft.

### BRIEF DESCRIPTION OF THE DRAWINGS

The accompanying drawings, which are incorporated herein and constitute part of this specification, illustrate the presently preferred embodiments of the present disclosure, and, together with the general description above and the detailed description given below, serve to explain the features of the present disclosure. In the drawings:

FIG. 1 is a side view of a cross balloon catheter having a perfusion-style balloon, according to an embodiment of the present disclosure.

FIG. 2 is a side view of a cross balloon catheter having a tapered perfusion-style balloon, according to an embodiment of the present disclosure.

FIG. 3 is a flow chart of a method for using a cross balloon catheter having a perfusion-style balloon, according to an embodiment of the present disclosure.

FIG. 4 is a side view of a cross balloon catheter having a perfusion-style balloon and a post-dilatory noncompliant balloon, according to an embodiment of the present disclosure.

FIG. 5 is a flow chart of a method for using a cross balloon catheter having a perfusion-style balloon and a post-dilatory noncompliant balloon, according to an embodiment of the present disclosure.

FIG. 6 is a side view of a cross balloon catheter having a perfusion-style balloon and a plurality of wire lumens, according to an embodiment of the present disclosure.

### DETAILED DESCRIPTION

The present disclosure will now be described more fully hereinafter. However, many modifications and other embodiments of the present disclosure set forth herein will come to mind to one skilled in the art to which the present disclosure pertains having the benefit of the teachings presented in the foregoing descriptions. Therefore, it is to be understood that the present disclosure is not to be limited to the specific embodiments disclosed and that modifications and other embodiments are intended to be included within the scope of the appended claims.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout the specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the disclosure.

Reference throughout this specification to “one embodiment,” “an embodiment,” or similar language means that a particular feature, structure, or characteristic described in connection with the indicated embodiment is included in at least one embodiment of the present disclosure. Thus, the phrases “in one embodiment,” “in an embodiment,” “in some embodiments,” and similar language throughout this specification may, but do not necessarily, all refer to the same embodiment.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to.”

When used in this document, the term “exemplary” is intended to mean “by way of example” and is not intended to indicate that a particular exemplary item is preferred or required.

As used herein, the terms “about” and “approximately” may be used interchangeably and is meant to encompass variations of ±20%, ±10%, ±5%, ±1%, and ±0.1% from the specified value, as such variations are appropriate.

As used herein, the term “communicate” and “communication” include, but are not limited to, the connection of fluid system elements, either directly or remotely, enabling fluid interface among and between said elements.

As used herein, the term “connectable” or “connection” refers to being able to be joined together for purposes including, but not limited to, allowing a flow of fluid. The term “connectable” can refer to being able to be joined together temporarily or permanently.

As used herein, a “patient” or “subject” is a member of any animal species, preferably a mammalian species, optionally a human. The subject can be an apparently healthy individual, an individual suffering from a disease, or an individual being treated for a disease.

As used herein, the terms “treating” and “treatment” refer to the management and care of a patient having a pathology or condition by administration of one or more therapies contemplated by the present disclosure. Treating also includes administering one or more methods of the present disclosure or using any of the systems, devices, or compositions of the present disclosure in the treatment of a patient. As used herein, “treatment” or “therapy” refers to both therapeutic treatment and prophylactic or preventative measures. “Treating” or “treatment” does not require complete alleviation of signs or symptoms, does not require a cure, and includes protocols having only a marginal or incomplete effect on a patient.

As used herein, the term “vessel” refers to a bodily passage or tract through which a catheter of the present disclosure may be disposed. This may include, e.g., blood vessels, arteries, veins within the circulatory system, the digestive tract, urinary tract, biliary tract, body cavities, or other passages in the body.

The present disclosure relates to perfusion dilatation catheters and methods of use. According to various embodiments, the present disclosure provides technology that enables a more simplified and reproducible approach to 1) verify a location of a physicians' equipment in relation to an intended area (e.g., a bioprosthetic aortic valve and its stent frame), and 2) obtain coaxial alignment of the physicians' equipment to allow safe and reliable movement (e.g., crossing of the aortic valve into the left ventricle) of the catheter. This further aids in improving clinical outcomes for patients (e.g., prolonged procedural time, inability to cross the valve, potential increase in TIA or stroke, damage to prior bioprosthetic aortic valve device, dislodgement of aortic valve prosthesis leading to emergency open heart surgery or death, etc.).

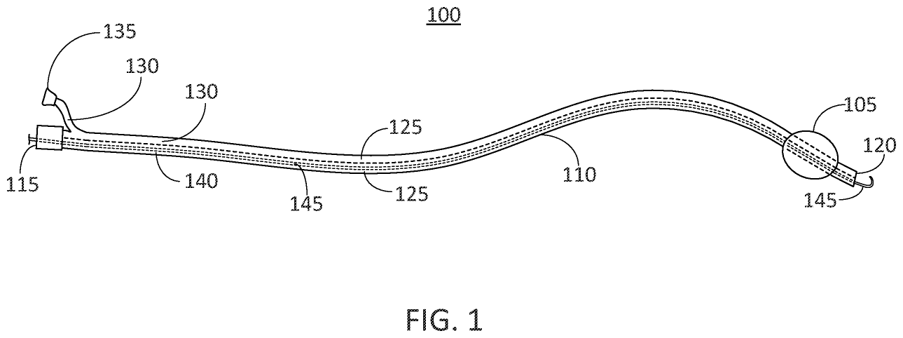

Referring now to FIG. 1, a side view of a cross balloon catheter 100 having a perfusion-style balloon 105 is illustratively depicted, in accordance with various embodiments of the present disclosure.

According to various embodiments, the cross balloon catheter 100 may comprise an 8-10 French large bore double-lumen catheter. The cross balloon catheter 100 may comprise an elongated catheter shaft 110. The elongated catheter shaft 110 may have a proximal end 115 and a distal end 120. According to various embodiments, the elongated catheter shaft 110 may be configured to enclose a plurality of lumens 125. The plurality of lumens 125 may comprise one or more inflation lumens 130. According to various embodiments, the one or more inflation lumens may be configured to enable at least one inflation medium to be transported to the perfusion-style balloon 105. According to various embodiments, the cross balloon catheter 100 may comprise one or more balloon inflation/deflation ports 135. According to various embodiments, the one or more balloon inflation/deflation ports 135 may be configured to supply the at least one inflation medium to the perfusion-style balloon 105 for inflating the perfusion-style balloon 105. According to various embodiments, the one or more balloon inflation/deflation ports 135 may be configured to remove the at least one inflation medium from the perfusion-style balloon 105 for deflating the perfusion-style balloon 105.

According to various embodiments, at least one of the plurality of lumens 125 may comprise a guidewire-receiving lumen 140. The guidewire-receiving lumen 140 may be configured to receive a guidewire 145 (e.g., a curved-tipped guidewire such as a J-wire).

According to various embodiments, the guidewire may comprise a 0.035 inch guidewire. It is noted, however, that other suitable diameter guidewires may be incorporated while maintaining the spirit and functionality of the present disclosure. According to various embodiments, the guidewire-receiving lumen 140 may be configured to receive one or more wires (e.g., guidewires 145) through the elongated catheter shaft 110 from the proximal end 115 of the elongated catheter shaft 110 to the distal end 120 of the elongated catheter shaft 110.

According to various embodiments, the perfusion-style balloon 105 may be positioned along an outer surface of the elongated catheter shaft 110. According to various embodiments, the perfusion-style balloon 105 may be positioned at or near the distal end 120 of the elongated shaft. According to various embodiments, the perfusion-style balloon 105 may be positioned approximately 8-10 mm from the distal end 120 of the elongated catheter shaft 110. It is noted, however, that other positions of the perfusion-style balloon 105 may be incorporated while maintaining the spirit and functionality of the present disclosure.

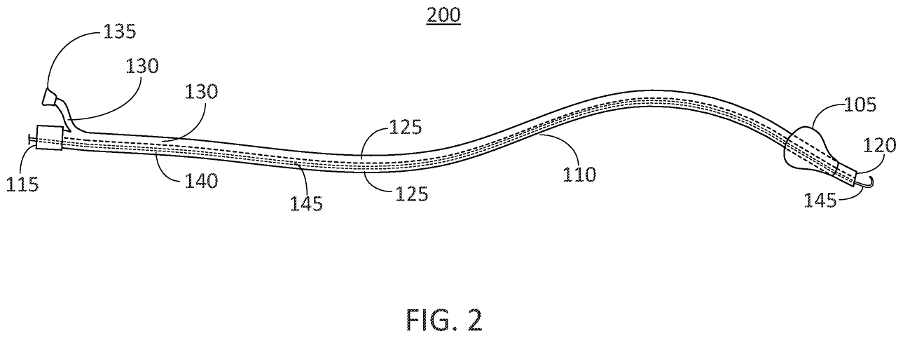

According to various embodiments, the perfusion-style balloon 105 may have an inflated diameter of approximately 15-25 mm. It is noted, however, that other suitable diameters may be implemented while maintaining the spirit and functionality of the present disclosure. According to various embodiments, the perfusion-style balloon 105 may have a taper. According to various embodiments, the perfusion-style balloon 105 may be tapered toward the distal end 120 of the elongated catheter shaft 110, such as shown, e.g., in the example cross balloon catheter 200 illustrated in FIG. 2.

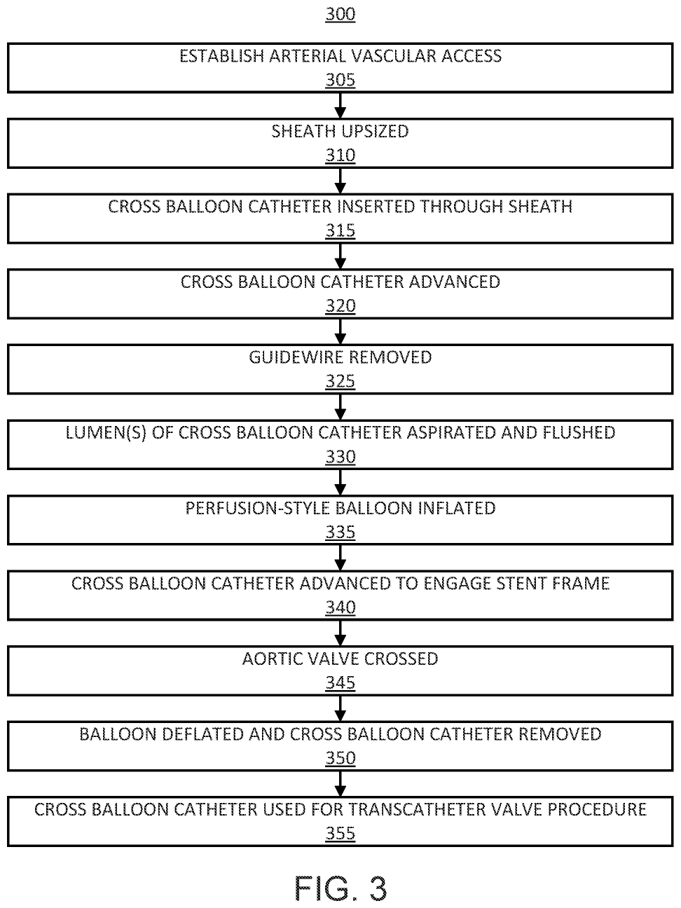

Referring now to FIG. 3, an example method 300 of using a cross balloon catheter (e.g., cross balloon catheters 100, 200 of FIGS. 1-2) during a structural heart procedure is illustratively depicted, in accordance with various embodiments of the present disclosure.

At 305, the treating physician(s) establish(es) arterial vascular (e.g., femoral, brachial, radial, etc.) access with a short vascular access sheath. This occurs at the beginning of a planned structural heart intervention procedure. Once access is established, the sheath, at 310, may be upsized to a standard large-bore sheath used, e.g., for TAVR or Valve-in-Valve TAVR procedures.

Over a standard J-shaped guidewire through the primary lumen of the balloon catheter, the cross balloon catheter, at 315, may be inserted through the above-mentioned vascular access sheath. The cross balloon catheter, at 320, is advanced, e.g., under fluoroscopic guidance, toward a proximal portion of the aortic root. At 325, a guidewire (e.g., a standard J-guidewire) is removed from the cross balloon catheter and, at 330, one of the one or more lumens of the cross balloon catheter may be aspirated and/or flushed with heparinized saline. According to various embodiments, the cross balloon catheter may comprise a double-lumen catheter. It is noted, however, that the cross balloon catheter may comprise other suitable numbers of lumens (e.g. a triple-lumen catheter), while maintaining the spirit and functionality of the present disclosure.

According to various embodiments, under fluoroscopic guidance, at 335, the perfusion-style balloon is inflated and, at 340, the cross balloon catheter is advanced forward to engage a top of a bioprosthetic aortic valve's stent frame.

At 345, once the perfusion balloon is inflated and engaged with the stent frame (and insured not “outside the frame”), using standard techniques, the aortic valve is crossed with exchange length straight-wires and/or the use of one or more catheters (e.g., 6-French catheters) via a medium bore lumen of the cross balloon catheter. According to various embodiments, engaging the stent frame includes confirming that the perfusion balloon is not positioned outside the stent frame.

At 350, once the wire has crossed the valve, the perfusion balloon is deflated and the cross balloon catheter is removed off the wire. According to various embodiments, at 355, the physician(s) proceed with standard protocol of the cross balloon catheter being used for the transcatheter valve procedure.

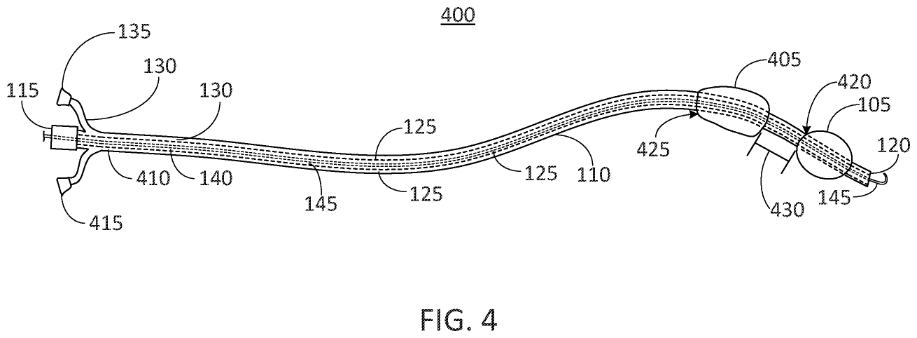

Referring now to FIG. 4, a side view of a cross balloon catheter 400 comprising a perfusion-style balloon 105 and a post-dilatory noncompliant balloon 405 is illustratively depicted, in accordance with various embodiments of the present disclosure.

According to various embodiments, the cross balloon catheter 400 may comprise an 8-10 French large bore double-lumen catheter. According to various embodiments, the cross balloon catheter 400 may comprise a triple-lumen catheter (e.g., lumens 130, 140, and 410, as shown, e.g., in FIG. 4).

The cross balloon catheter 400 may comprise an elongated catheter shaft 110. The elongated catheter shaft 110 may have a proximal end 115 and a distal end 120. According to various embodiments, positioned along the elongated catheter shaft 110 may be the perfusion-style balloon (a distal balloon) 105 and the post-dilatory noncompliant balloon (a proximal balloon) 405. It is noted, however, that, according to some embodiments, the distal balloon may comprise a perfusion-style balloon, post-dilatory noncompliant balloon, and/or other suitable type of balloon, and/or the proximal balloon may comprise a perfusion-style balloon, post-dilatory noncompliant balloon, and/or other suitable type of balloon.

According to various embodiments, the elongated catheter shaft 110 may be configured to enclose a plurality of lumens 125. The plurality of lumens 125 may comprise an inflation and/or deflation lumen 130 configured to transport at least one inflation medium to the perfusion-style balloon 105 and/or remove at least one inflation medium from the perfusion-style balloon 105, and an inflation/deflation lumen 410 configured to transport at least one inflation medium to the post-dilatory noncompliant balloon 405 and/or remove at least one inflation medium from the post-dilatory noncompliant balloon 405. According to various embodiments, the cross balloon catheter 100 includes at least one inflation port. According to various embodiments, the at least one inflation port may comprise a proximal balloon inflation/deflation port 415 configured to supply the at least one inflation medium for inflating the perfusion-style balloon 105 and a distal balloon inflation port 135 configured to supply the at least one inflation medium for inflating the post-dilatory noncompliant balloon 405. The perfusion-style balloon 105 and inflation lumen 130 may form a distal balloon system 420, and the post-dilatory noncompliant balloon 405 and inflation lumen 410 may form a proximal balloon system 425.

According to various embodiments, the perfusion-style balloon 105 and the post-dilatory noncompliant balloon 405 may be affixed to an outer surface of the elongated catheter shaft 110, separated by a set distance 430. According to various embodiments, the set distance may be approximately 10-15 mm. It is noted, however, that other suitable set distances may be utilized, while maintaining the spirit and functionality of the present disclosure.

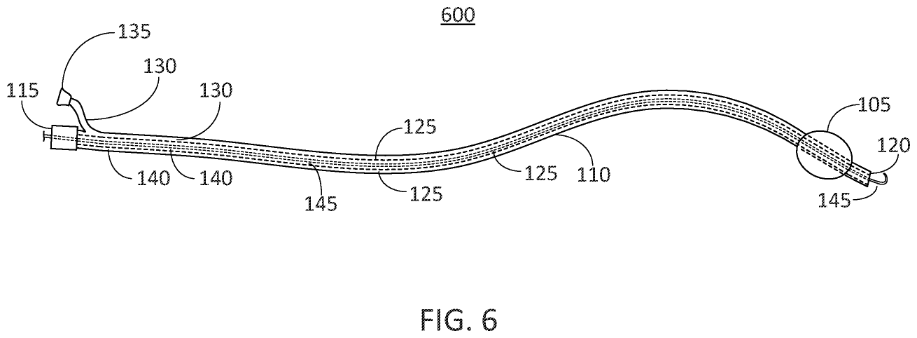

According to various embodiments, at least one of the plurality of lumens 125 may comprise a guidewire-receiving lumen 140 configured to receive a guidewire 145 (e.g., a curved-tipped guidewire such as a J-wire). The guidewire-receiving lumen 140 may be configured to receive one or more wires (e.g., guidewires 145) through the elongated catheter shaft 110 from the proximal end 115 of the elongated catheter shaft 110 to the distal end 120 of the elongated catheter shaft 110. According to various embodiments, the at least one of the plurality of lumens 125 may comprise a plurality of lumens 140 configured to receive wire through the elongated catheter shaft 110 to the distal end 120 of the elongated catheter shaft 110, as shown, e.g., in cross balloon catheter 600 in FIG. 6.

According to an exemplary embodiment, the perfusion-style balloon 105 may be positioned along an outer surface of the elongated catheter shaft 110. According to various embodiments, the perfusion-style balloon 105 may be positioned at or near the distal end 120 of the elongated shaft. According to various embodiments, the perfusion-style balloon 105 may comprise a proximal end and a distal end. The distal end may be positioned approximately 8-10 mm from the distal end 120 of the elongated catheter shaft 110. It is noted, however, that other suitable positions of the distal end of the perfusion-style balloon 105 in relation to the distal end 120 of the elongated catheter shaft 110 may be incorporated, while maintaining the spirit and functionality of the present disclosure. According to various embodiments, the perfusion-style balloon 105 may have an inflated diameter of approximately 15-25 mm. According to an exemplary embodiment, the perfusion-style balloon 105 may have an inflated diameter of approximately 15-20 mm. It is noted, however, that other suitable diameters may be incorporated while maintaining the spirit and functionality of the present disclosure. According to various embodiments, the perfusion-style balloon 105 may have a taper. According to various embodiments, the perfusion-style balloon 105 may be tapered toward the distal end 120 of the elongated catheter shaft 110.

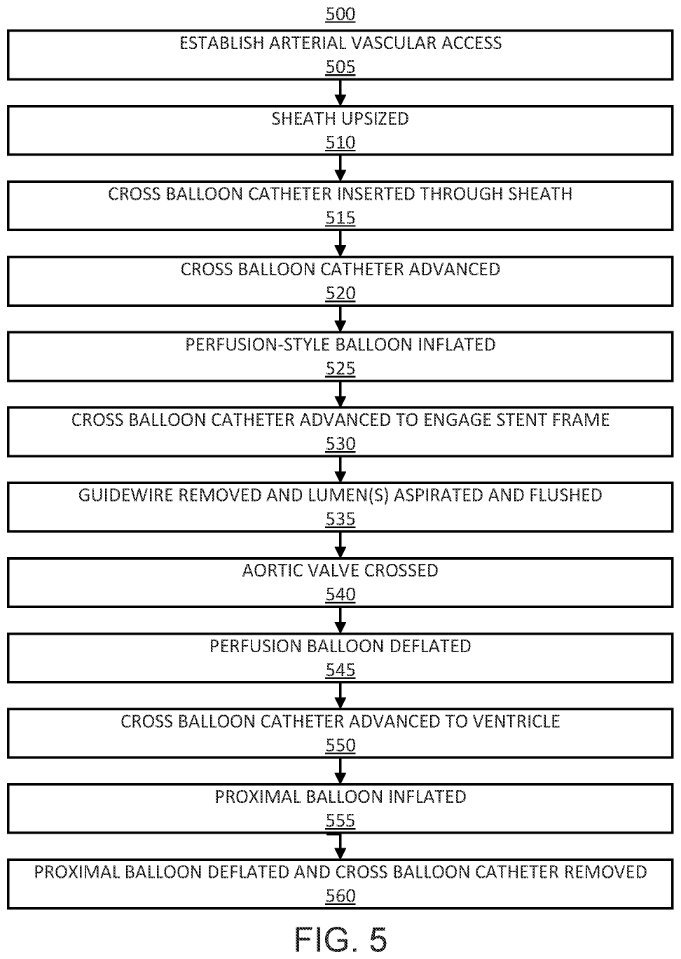

Referring now to FIG. 5, an example method 500 of using a cross balloon catheter (e.g., cross balloon catheters 400 of FIG. 4) during a structural heart procedure is illustratively depicted, in accordance with various embodiments of the present disclosure.

At 505, the treating physician(s) establish(es) arterial vascular (e.g., femoral, brachial, radial, etc.) access with a short vascular access sheath. This occurs at the beginning of a planned structural heart intervention procedure. Once access is established, the sheath, at 510, is upsized to a standard large-bore sheath used, e.g., for TAVR or Valve-in-Valve TAVR procedures.

Over a standard J-shaped guidewire through the primary lumen of the balloon catheter, the cross balloon catheter, at 515, is inserted through the above-mentioned vascular access sheath. The cross balloon catheter, at 520, is advanced, under fluoroscopic guidance, toward a proximal portion of the aortic root. At 525, under fluoroscopic guidance, the perfusion-style balloon is inflated and, at 530, the cross balloon catheter is advanced forward to engage a top of a bioprosthetic aortic valve's stent frame.

At 535, once the perfusion balloon is inflated and engaged with the stent frame (and insured not “outside the frame”), using standard techniques, guidewire (e.g., a standard J-guidewire) is removed from the cross balloon catheter and one of the one or more lumens of the cross balloon catheter is aspirated and flushed with heparinized saline.

According to various embodiments, under fluoroscopic guidance, at 540, once the perfusion balloon is inflated and engaged with the stent frame (and insured not “outside the frame”), using standard techniques, the aortic valve is crossed with exchange length straight-wires and/or the use of one or more catheters (e.g., 6-French catheters) via a medium bore lumen of the cross balloon catheter. According to various embodiments, engaging the stent frame includes confirming that the perfusion balloon is not positioned outside the stent frame.

At 545, once the wire has crossed the aortic valve, the perfusion balloon is deflated and, at 550, the cross balloon catheter is advanced into the left ventricle so as to align the proximal noncompliant balloon to be at the level of the aortic valve. At this time, at 555, the proximal balloon is inflated via standard practice to optimize valve performance. The proximal balloon, at 560, is then deflated and the cross balloon catheter is removed from the body over the guidewire in place.

Methods of the present disclosure may be employed on any suitable vessel including, but not limited to, the inferior vena cava, the superior vena cava, the iliac veins, the aorta, the pulmonary artery, cardiac artery, or the pulmonary vein.

In this document, when terms such “first” and “second” are used to modify a noun, such use is simply intended to distinguish one item from another, and is not intended to require a sequential order unless specifically stated. The term “approximately,” when used in connection with a numeric value, is intended to include values that are close to, but not exactly, the number. For example, in some embodiments, the term “approximately” may include values that are within +/−10 percent of the value.

When used in this document, terms such as “top” and “bottom,” “upper” and “lower,” or “front” and “rear,” are not intended to have absolute orientations but are instead intended to describe relative positions of various components with respect to each other. For example, a first component may be an “upper” component and a second component may be a “lower” component when a device of which the components are a part is oriented in a first direction. The relative orientations of the components may be reversed, or the components may be on the same plane, if the orientation of the structure that contains the components is changed. The claims are intended to include all orientations of a device containing such components.

The features and functions described above, as well as alternatives, may be combined into many other different systems or applications. Various alternatives, modifications, variations or improvements may be made by those skilled in the art, each of which is also intended to be encompassed by the disclosed embodiments.

It will be apparent to one of ordinary skill in the art that various combinations and/or modifications and variations can be made in the perfusion dilatation catheter systems and devices of the present disclosure depending upon the specific needs for operation and as dictated by the therapeutic needs of the patient. Moreover, features illustrated or described as being part of one embodiment may be used on another embodiment to yield a still further embodiment.

1. A perfusion dilatation catheter, comprising:
an elongated catheter shaft, comprising a proximal end and a distal end, configured to:
enclose a plurality of lumens, comprising:
a proximal balloon inflation lumen; and
a distal balloon inflation lumen; and
house at least one inflation port configured to supply at least one inflation medium through the proximal balloon inflation lumen and the distal balloon inflation lumen;
a proximal balloon system, comprising:
a proximal balloon positioned along the elongated catheter shaft; and
the proximal balloon inflation lumen; and
a distal balloon system, comprising:
a distal balloon positioned along the elongated catheter shaft, distal to the proximal balloon; and
the distal balloon inflation lumen.

2. The perfusion dilatation catheter of claim 1, wherein the proximal balloon and the distal balloon:
are affixed to an outer surface of the elongated catheter shaft; and
are separated by a set distance.

3. The perfusion dilatation catheter of claim 2, wherein the set distance is approximately 10-15 mm.

4. The perfusion dilatation catheter of claim 1, wherein:
the at least one inflation port comprises a first inflation port and a second inflation port,
the first inflation port is configured to supply the at least one inflation medium through the proximal balloon inflation lumen to the proximal balloon, and
the second inflation port is configured to supply the at least one inflation medium through the distal balloon lumen to the distal balloon.

5. The perfusion dilatation catheter of claim 1, wherein the distal balloon comprises a perfusion-style balloon.

6. The perfusion dilatation catheter of claim 1, wherein the distal balloon is tapered toward the distal end of the elongated catheter shaft.

7. The perfusion dilatation catheter of claim 1, wherein the distal balloon has an inflated diameter of approximately 15-20 mm.

8. The perfusion dilatation catheter of claim 1, wherein the proximal balloon comprises a post-dilatory noncompliant balloon.

9. The perfusion dilatation catheter of claim 1, wherein the proximal balloon has an inflated diameter of approximately 15-20 mm.

10. The perfusion dilatation catheter of claim 1, wherein:
the distal balloon comprises a proximal end and a distal end, and
the distal end of the distal balloon is approximately 8-10 mm from the distal end of the elongated catheter shaft.

11. The perfusion dilatation catheter of claim 1, wherein the plurality of lumens comprises a lumen configured to receive a curved-tipped guidewire.

12. The perfusion dilatation catheter of claim 1, wherein:
the plurality of lumens comprises a plurality of wire lumens, and
each wire lumen, of the plurality of wire lumens, is configured to receive a wire through the elongated catheter shaft to the distal end of the elongated catheter shaft.

13. A perfusion dilatation catheter, comprising:
an elongated catheter shaft, comprising a proximal end and a distal end, configured to:
enclose a plurality of lumens, comprising:
a proximal balloon inflation lumen; and
a distal balloon inflation lumen; and
house at least one inflation port configured to supply at least one inflation medium through the proximal balloon inflation lumen and the distal balloon inflation lumen;
a distal balloon system, comprising:
a perfusion-style balloon positioned along the elongated catheter shaft and tapered toward the distal end of the elongated catheter shaft; and
the distal balloon inflation lumen; and
a proximal balloon system, comprising:
a post-dilatory noncompliant balloon positioned along the elongated catheter shaft,
distal to the perfusion-style balloon; and
the proximal balloon inflation lumen.

14. The perfusion dilatation catheter of claim 13, wherein the perfusion-style balloon and the post-dilatory noncompliant balloon:
are affixed to an outer surface of the elongated catheter shaft; and
are separated by a set distance.

15. The perfusion dilatation catheter of claim 14, wherein the set distance is approximately 10-15 mm.

16. The perfusion dilatation catheter of claim 13, wherein:
the at least one inflation port comprises a first inflation port and a second inflation port,
the first inflation port is configured to supply the at least one inflation medium through the proximal balloon inflation lumen to the post-dilatory noncompliant balloon, and
the second inflation port is configured to supply the at least one inflation medium through the distal balloon lumen to the perfusion-style balloon.

17. The perfusion dilatation catheter of claim 13, wherein the perfusion-style balloon is tapered toward the distal end of the elongated catheter shaft.

18. The perfusion dilatation catheter of claim 13, wherein:
the perfusion-style balloon has an inflated diameter of approximately 15-20 mm, and
the post-dilatory noncompliant balloon has an inflated diameter of approximately 15-20 mm.

19. The perfusion dilatation catheter of claim 13, wherein:
the perfusion-style balloon comprises a proximal end and a distal end, and
the distal end of the perfusion-style balloon is approximately 8-10 mm from the distal end of the elongated catheter shaft.

20. The perfusion dilatation catheter of claim 19, wherein the plurality of lumens comprises a plurality of wire lumens, each wire lumen being configured to receive a wire through the elongated catheter shaft to the distal end of the elongated catheter shaft.
